(19) United States
(12) Patent Application Publication
(10) Pub. No.: US 2026/0363573 A1
(43) Pub. Date: Nov. 19, 2026

(54) SYSTEM FOR IDENTIFYING RESPIRATORY SUFFERING FROM FACIAE IMAGING ON A PATIENT, AND METHOD USING SAID DEVICE

(71) Applicants: SORBONNE UNIVERSITE, Paris (FR); CENTRE NATIONAL DE LA RECHERCHE SCIENTIFIQUE, Paris (FR); INSTITUT NATIONAL DE LA SANTE ET DE LA RECHERCHE MEDICALE (I.N.S.E.R.M), Paris (FR); ASSISTANCE PUBLIQUE HOPITAUX DE PARIS (AP-HP), Paris (FR)

(72) Inventors: Thomas SIMILOWSKI, Paris (FR); Maxens DECAVELE, Paris (FR); Kevin BAILLY, Paris (FR); Gauthier TALLEC, Paris (FR)

(21) Appl. No.: 19/478,258

(22) PCT Filed: Apr. 24, 2023
(86) PCT No.: PCT/IB2023/000228

§ 371 Date: Oct. 23, 2025

(51) Int. Cl. A61B 5/08; A61B 5/00; A61B 5/01; A61B 5/145

(52) U.S. Cl. CPC A61B 5/0816; A61B 5/0077; A61B 5/015; A61B 5/14542

(57) ABSTRACT

A system is provided for monitoring respiratory suffering or discomfort on a patient. The system has an acquisition device including at least a video camera and configured to acquire an image stream of the face of said patient. The system configured to measure the respiratory rate of the patient and the cardiac rate of the patient.

The system being is configured to
detect on the image stream at least one predefined facial cue on the face of the patient associated to respiratory distress, an action score being attributed based on the presence or absence of said facial cue(s), and
attribute a respiratory score based on the measured value of the respiratory rate. The system is also configured to attribute a cardiac score based on the measured value of the cardiac rate, compute a global score, and, if the global score is greater than a predefined threshold, trigger an action, especially an alarm, to indicate respiratory distress.

### TECHNICAL FIELD

The invention relates to systems and methods for monitoring respiratory suffering or discomfort on patients, especially patients who have difficulties communicating with a caregiver, as it is for example the case of patients placed under artificial ventilation in an intensive care unit (ICU).

### PRIOR ART

Respiratory distress is a frequently encountered problem in critically ill patients placed on artificial ventilation. Even though the artificial respirator is supposed to relieve the respiratory distress of such patients, between 35% and 50% of intubated patients in intensive care who can verbally or gesturally communicate will declare that they are suffering from their breathing. The symptom that conveys such an experience of upsetting or distressing awareness of breathing is called dyspnea. Like pain, dyspnea is a multidimensional phenomenon. It associates one or several respiratory sensations (called lack of air, air hunger, smothering, suffocating, excessive breathing effort, chest constriction, etc.) to one of several emotions (like fear, anxiety, frustration, anger, etc.). Dyspnea is the symptom of respiratory-related brain suffering, an ensemble of brain reactions to abnormal messages from the respiratory system in particular. Dyspnea is one of the consequences of respiratory-related brain suffering, together with an increased ventilatory motor response (that can translate into accelerated breathing and physical signs of respiratory distress), a neurovegetative response testifying to a physiological stress (that can translate in tachycardia, hypertension, sweating, etc.) and various behavioral manifestations including a facial expression suggesting fear or effort.

In intubated and artificially ventilated critically ill patients, respiratory-related brain suffering, and the ensuing dyspnea are associated with adverse clinical consequences in the short, medium, and long term. In the short term, respiratory-related brain suffering, and the ensuing dyspnea causes a feeling of intense fear, the fear of dying. In the medium term, respiratory-related brain suffering is associated with a longer duration of artificial ventilation than in patients without dyspnea. In the long term, and echoing the immediate psychological consequences, respiratory-related brain suffering and the ensuing dyspnea, in particular when occurring as repeated episodes during a stay in intensive care, is independently associated with the occurrence of a post-traumatic stress disorder.

Insofar as the mission of a caregiver is above all to relieve suffering, the relief of respiratory-related brain suffering and the ensuing dyspnea is listed among the priority care goals, as written in the public health code, article 1110-5 version of Feb. 4, 2016: “Every person has, . . . the right to receive, . . . the best possible relief of suffering”.

In order to be treated, respiratory-related brain suffering must first be detected. Dyspnea is its most straightforward manifestation, but by definition its assessment relies on the self-report made by the patient. Yet, more than 50% of intubated patients in the ICU are unable to report their dyspnea because of their strongly altered self-assessment and/or communication capacities. These non-communicative patients are however exposed to the consequences of respiratory-related brain suffering in exactly the same way as communicative patients, and the impossibility to communicate their suffering worsens the corresponding traumatic impact. It is of the utmost importance to understand that the inability to verbally or gesturally communicate (in other words, the impossibility to report dyspnea) does not exclude the possibility that an individual experience respiratory-related brain suffering and requires appropriate management to relieve it. Moreover, there are effective therapeutic means to effectively relieve dyspnea such as optimizing ventilator settings or administering opiates.

Besides, it is unfortunately shown that caregivers underestimate the suffering of an intubated patient when they are asked to estimate its intensity just by looking at the patient, without asking him. Thus, caregivers are a priori unable to reliably estimate the intensity of respiratory-related brain suffering that an intubated patient may present at a given time with a simple judgment or intuition. In addition, the presence of caregivers at the bedside of patients is by nature intermittent.

There is therefore a need for tools to detect or rather to infer or strongly suspect respiratory-related brain suffering in non-communicative intubated patients, who cannot self-report dyspnea.

There are very few such tools available in general, and in intubated patients undergoing artificial ventilation in particular.

An observational scale, the “Mechanical Ventilation-Respiratory Distress Observation Scale (MV-RDOS)” has been proposed as an alternative to self-report of dyspnea for non-communicating intubated patients (Decavèle et al. Eur Respir J, 2018, Decavèle et al. Am J Respir Crit Care Med, 2023). As an example, an MV-RDOS value≥2.6 predicts a VAS-D>30 mm with a specificity of more than 90% (AUC 0.78). However, observation scales require calculation time and human resources, like a nurse or a doctor. The information they provide is discontinuous, whereas dyspnea can occur at any time, and therefore possibly when no caregiver is in the room. Finally, observation scales rely in part on the identification of the facial expression of fear or of abdominal paradox, of which the assessment remains operator-dependent and complex. Moreover, MV-RDOS is not yet strictly validated in non-communicative patients and is therefore only used in clinical research in its current state of development.

Two other tools proposed to identify respiratory-related brain suffering when dyspnea is not available are neurophysiological biomarkers, namely: 1) surface electromyographic (EMG) recordings of the diaphragm and extra-diaphragmatic inspiratory muscles (parasternal intercostals, Alae Nasi, Scalene); and 2) electroencephalographic (EEG) respiratory-related signatures, including, but not limited to, pre-inspiratory potentials detected by before inspiration and attesting to the cortical preparation of the inspiratory movement. However, these tools are reserved for specialized research teams, are complex to set up, especially in the ICU patient because of technical difficulties arising from agitation, sweat, and any artifacts related to the contact with the patient. They also require a a posteriori data processing, thus not giving any indication in real time on the probability of dyspnea of a patient.

The inventors have obtained data in healthy participants subjected to laboratory-induced dyspnea (also called experimental dyspnea) showing that certain facial action units (AU) defined in the Facial Action Coding System (FACS) are activated in these circumstances. This results in patterns of facial expression that can be distinguished from those of pain, according to the nature of the involved AUs, and the frequency and intensity of their activation, and particularly in evocative clinical or experimental contexts.

The facial action unit AU4, corresponding to a frown, is strongly correlated with experimental dyspnea of the excessive effort type, as can be provoked by inspiratory resistive load. AU1, corresponding to an inner eyebrow raise, AU2, corresponding to an outer eyebrow raise, and AU5, corresponding to an eyelid widening, are strongly correlated with experimental air hunger type dyspnea, as can be provoked by hypercapnia. AUs 1, 2, 4 and 5 are therefore of interest to monitor/detect to infer respiratory-related brain suffering in a patient. These AUs are located on the upper face which is of major interest in intubated patients in whom the lower face may be at least partially masked by the intubation tube and the circuit and/or filter of the artificial ventilator. Algorithms for automated recognition of AUs 1, 2, 4 and 5 on videos obtained by RVB cameras are known.

There is no known automated facial monitoring of respiratory distress in the ICU or in any other field that combines the measurement of AUs 1, 2, 4, 5, with physiological variables such as heart rate, respiratory rate or oxygen saturation. Yet algorithms are known for the determination of such variables from RVB cameras, as explained below.

There are no data on the possibilities of extracting the respiratory rate from the thermal capture carried out at the level of the ventilation circuit of critically ill intubated patients placed under artificial ventilation. Yet algorithms are known for the determination of respiratory rate from thermal capture in subjects breathing naturally, as explained below.

Facial expression recognition algorithms exist but, for the most part, these algorithms are based on visible capture and therefore not applicable at night in patients, while such patients can suffer from dyspnea during the day as well as at night.

Some algorithms are known for measuring the heart rate by visible camera measuring the color differences (RGB) at the level of the face, especially the cheeks, related to the heartbeat or by thermal capture measuring the temperature variations on the surface of the face related to the heartbeat, as the blood that reaches the face during a heartbeat warms the surface of the skin of the face, for a few tenths of a degree of Celsius, which is perceptible by thermal camera.

There are algorithms for measuring the respiratory rate from thermal video acquisition at the nostrils and/or mouth in far or medium infrared.

The articles of Garbey M et al., “Contact-free measurement of cardiac pulse based on the analysis of thermal imagery”, IEEE Trans Biomed Eng. 2007; 54 (8): 1418-26, and of Manullang M C T, Lin Y H, Lai S J, Chou N K, “Implementation of Thermal Camera for Non-Contact Physiological Measurement: A Systematic Review, Sensors (Basel). 2021 Nov. 23; 21 (23): 7777, relate to heart rate measurement by thermal camera.

Methods for measuring the respiratory rate by thermal camera are for example described in Pereira C B, et al. “Remote monitoring of breathing dynamics using infrared thermography”, Biomed Opt Express. 2015; 6:4378-94, and Procházka A, Charvátová H, Vyšata O, Kopal J, Chambers J., “Breathing Analysis Using Thermal and Depth Imaging Camera Video Records”, Sensors (Basel). 2017; 17:1408.

The article of Sikka K, Ahmed A A, Diaz D, Goodwin M S, Craig K D, Bartlett M S, Huang J S. “Automated Assessment of Children's Postoperative Pain Using Computer Vision”, Pediatrics. 2015 July; 136 (1): e124-31, relates to FACS facial expression recognition by artificial intelligence and visible camera in children.

The article of Ko B C., “A Brief Review of Facial Emotion Recognition Based on Visual Information”, Sensors (Basel), 2018 Jan. 30; 18 (2): 401, relates to automated facial expression measurement using a visible camera.

Automated FACS measurement by thermal camera are notably described in Rodríguez Medina D A, et al., “Biopsychosocial Assessment of Pain with Thermal Imaging of Emotional Facial Expression in Breast Cancer Survivors”, Medicines (Basel). 2018 Mar. 30; 5 (2): 30, in Wang S. et al., “Thermal Augmented Expression Recognition”, IEEE Trans Cybern. 2018 July; 48 (7): 2203-2214, and in Jiang G. et al., “Facial expression recognition using thermal image”, Conf Proc IEEE Eng Med Biol Soc. 2005; 2006:631-3.

The technology called Quantiq measures heart rate, respiratory rate and oxygen saturation (SpO2) by analyzing the patient's face with a smartphone camera.

The tool developed by Samdoc monitors pain facial expressions by infrared/thermal camera.

The “Smart cocoon” solution developed by Valeo proposes a monitoring of the heart and respiratory rate from the analysis of the face, especially in order to detect signs of fatigue while driving, as for example eyelid closure.

The smartphone application “Lucine” helps to measure, analyze and relieve pain at home with facial recognition of pain from a smartphone camera.

A similar project about facial recognition of pain is presented in an online conference at the following link: https://www.youtube.com/watch?v=j3OeF-IHItM.

Patent application US 2022/0160296 concerns the identification of the facial expression of pain and discloses a system including a wearable facial expression capturing system that is placed over a subject's face. The system is embedded with a plurality of sensors configured to detect biosignals from facial muscles and additionally includes a sensor node that recognizes facial expressions based on the detected biosignals. Pain experienced by the subject is assessed based on the facial expressions in conjunction with physiological signals obtained by other wearable sensors.

International application WO 2021/195138 describes a system receiving various physiological as well as physical information concerning a patient, and operational data from a ventilation device and medication delivery device, and providing the physiological and physical information, together with the operational data, to a neural network configured to analyze the information and data in order notably to assess the pain level of the patient.

Patent application US 2021/0052215 discloses a computer-based method for generating a current pain assessment of a neonate using facial expressions along with crying sounds, body movement, and vital signs changes and for using the current pain objective assessment to predict future pain objective assessment and assign a future pain probability score by incorporation spatiotemporal data into the multimodal assessment.

Patent application US 2019/0189259 provides a system for generating an optimized treatment experience for a patient. Patient experience data is captured, corresponding to a patient experience factor for the patient, which current level is determined based on the patient experience data, a customized therapy for the patient being generated based on the current level of the patient experience factor.

Patent application US 2018/0008360 relates to a system for remotely monitoring the health of a patient in a real-time, continuous manner, remotely delivering therapeutic medications to the patient, and facilitating communication between the patient and a remotely located patient care provider. The system uses a patient monitoring sensor configured to interface with a patient, to sense a patient vital sign and to transmit a signal representing the patient vital sign to the processor via a data communication subsystem.

International application WO 2011/017778 describes an anesthesia and consciousness depth monitoring system, using the combination of a biological signal evoked as a result of patient stimulation presented to a biological subject and a non-linear analysis method capable of capturing temporal changes in signal order or regularity in order to generate a measure indicative of said patient's level of anesthesia and consciousness depth.

There is a need to further improve the detection of respiratory suffering or discomfort in patients placed on artificial ventilation in the ICU, without resorting to verbal or non-verbal human interactions.

There is a need for such detection tool that also detect respiratory or discomfort in patients placed on artificial ventilation in the ICU during night where lighting conditions are lower than during the day and where visible signal-based devices are not usable.

The present invention notably seeks to meet this need.

### DISCLOSURE OF THE INVENTION

One subject of the invention is a system for monitoring respiratory suffering or discomfort on a patient, especially placed under artificial ventilation in an intensive care unit, the system comprising an acquisition device including at least a video camera and configured to acquire an image stream of the face of said patient, the system being configured to measure the respiratory rate of said patient, the cardiac rate of said patient, and optionally the transcutaneous oxygen saturation of said patient, the system being configured to:
detect on said image stream at least one predefined facial cue on the face of said patient associated to respiratory distress, an action score being attributed based on the presence or absence of said facial cue(s),
attribute a respiratory score based on the measured value of the respiratory rate, attribute a cardiac score based on the measured value of the cardiac rate,
optionally, attribute an oxygenation score based on the measured value of transcutaneous oxygen saturation,
compute a global score by adding said action, respiratory and cardiac scores, and
if said global score is greater than a predefined threshold, trigger an action, especially an alarm, to indicate respiratory distress.

The method according to the invention provides an automated, continuous, multidimensional, non-contact measurement of respiratory distress in humans by visible/thermal camera, based at least on the integration of facial expression analysis and the measure of respiratory and cardiac rates.

One of the advantages of this invention is that it provides an objective measurement whereas, in the known methods, the analysis of facial expression for detecting fear is not reliable when it is evaluated by the caregivers.

Another advantage of this invention, contrary to observational dyspnea scales, is the continuity of its measurement. Dyspnea occurs at any time, without warning, and caregivers are not necessarily in the patient's room at the time of dyspnea.

The invention allows increasing the number of efficient passages of the caregivers in the rooms of dyspnea patients. This could help filing a checklist for caregivers looking for risk factors of dyspnea in patients and trigger attempts to correct the possible identified etiological factors of dyspnea.

In a preferred embodiment, said predefined facial cues are facial action units, chosen among frowning, internal raising of the eyebrows, external raising of the eyebrows, and widening of the eyelids. Said action units (AU) corresponds respectively to AU 1, 2, 4, and 5 of the Facial Action Coding System (FACS).

Said patient being placed under artificial ventilation in an intensive care unit, the system may be configured so that the values of the respiratory rate are obtained from measures of the temperature variations of the artificial ventilation circuit.

The system of the invention is advantageously placed at a distance from the face of the patient equal or smaller than one meter.

### Camera

Said acquisition device may further include a thermal camera.

The system of the invention may be configured so that the values of the cardiac rate are obtained from measures of temperature variations on the surface of the face of the patient, especially the cheeks or forehead.

The image stream from the thermal camera and the image stream from the video camera are advantageously merged.

Said thermal camera may have a spectral range comprised between 7.5 μm and 14 μm.

Said thermal camera may have an optimal thermal sensitivity equal to 50 mK at 30 °C.

### Learning Module

The system of the invention may further include at least one learning module, trained beforehand to learn reference facial cues on previously acquired image streams from patients, said learning module being configured to detect said facial cues on the image stream from the patient under monitoring.

In a preferred embodiment, the face of the patient is detected in the image stream using a standard detection method, especially the Viola-Jones object detection framework, and each face image of the image stream is cropped and optionally aligned using facial landmark localization. Said learning module, notably a deep neural network or any other supervised machine learning based algorithm, is advantageously trained to predict the desired facial cues on an annotated database.

Said learning module may comprise, as described in patent application EP 21 306638.4, at least one recurrent network having:
task-specific cells, respectively blocks of tasks,
a differentiable order selector for determining a convex combination of a number M of different possible task orders, respectively blocks orders, for processing an input, by allocating a selector order coefficient Ti to each task order, respectively block order, and,
a merging module for computing the weighted average of the outputs given by the recurrent network for the M orders using as weights the order selector coefficients (π₁, π₂, . . . , π_{M}), the task-specific cells and the order selector being configured to trained jointly to minimize a loss function.

Such a set-up allows obtaining a very accurate detection of the AUs.

### Scores

In a preferred embodiment, the measurement of the respiratory and cardiac rates, and, optionally, the measurement of oxygen saturation, is averaged over a period of 10 seconds, giving six values per minute. This allows for a very regular follow-up but still allows time to observe variations. Said respiratory and cardiac scores are advantageously computed by summing the mean values of the measured respiratory and cardiac rates and by adding one point to the action score each time a facial cue is detected.

### Table 1 Below Shows Examples of Scores Calculated According to the Invention

TABLE 1

| | |
|---|---|
| Respiratory rate (cycles/min) | |
| ≤18 | 0 point |
| 19-30 | 1 point |
| >30 | 2 points |
| Cardiac rate (beats/min) | |
| <90 | 0 point |
| 90-109 | 1 point |
| ≥110 | 2 points |
| Oxygen saturation | |
| ≥90% | 0 point |
| <90 | 1 point |
| Action unit 1 or 2* | * If AU 1 and 2 are both |
| | present, only score 1 point |
| If absent | 0 point |
| If present | 1 point |
| Action unit 4 | |
| If absent | 0 point |
| If present | 1 point |
| Action unit 5 | |
| If absent | 0 point |
| If present | 1 point |

The respiratory score may be obtained automatically by summing each 10 seconds the different items listed in Table 1. An action may be triggered from a predefined threshold equal to or greater than 4.

In a variant, the heart rate and respiratory rate are used in their absolute values.

A threshold for the action score may be defined by qualifying a facial cue as present or absent. A facial cue may be given by the algorithms in probability of presence going from 0 (null probability) to 1 (certain probability). Said threshold may be equal to 0.5.

### Method

According to another one of its aspects, the invention also relates to a method for monitoring respiratory suffering or discomfort on a patient, especially placed under artificial ventilation in an intensive care unit, using a system comprising an acquisition device including at least a thermal camera and configured to acquire an image stream of the face of said patient, the system being configured to measure the respiratory rate of said patient, and to measure the cardiac rate of said patient,
the method comprising at least the steps of:
   detecting on said image stream at least one predefined facial cue on the face of said patient associated to respiratory distress, an action score being attributed based on the presence or absence of said facial cue(s),
   attributing a respiratory score based on the measured value of the respiratory rate,
   attributing a cardiac score based on the measured value of the cardiac rate,
   computing a global score by adding said action, respiratory and cardiac scores, and
   if said global score is greater than a predefined threshold, triggering an action, especially an alarm, to indicate respiratory distress.

The method advantageously uses at least one learning module, trained beforehand to learn reference facial cues on previously acquired image streams from patients, the method comprising the step of using said learning module to detect said facial cues on the image stream from the patient under monitoring.

The features described for the system applies to the method and vice and versa.

### Computer Program

According to another one of its aspects, the invention also relates to a computer program comprising instructions which, when the program is executed on a computer, cause the computer to carry out the steps of the method according to the invention and previously described.

The features described for the method applies to the computer program and vice and versa.

### BRIEF DESCRIPTION OF THE DRAWINGS

The invention may be better understood upon reading the following detailed description of non-limiting implementation examples thereof and on studying the appended drawing, in which:

FIG. 1 is a diagram illustrating some elements and some steps of an example of use of the invention.

### DETAILED DESCRIPTION

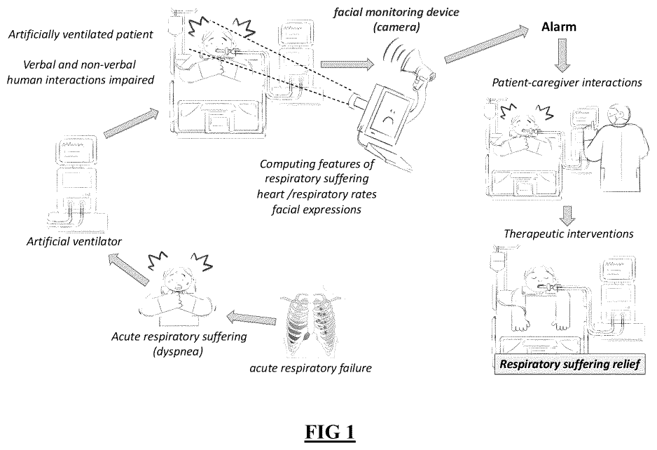

FIG. 1 illustrates an example of use of the invention, in the case of patient placed under artificial ventilation in an intensive care unit, with their verbal and non-verbal human interactions impaired, and suffering from acute respiratory failure and most probably from acute respiratory suffering, called dyspnea.

In this embodiment, and as previously described, a system is used, this system comprising a facial monitoring device including at least a video camera and configured to acquire an image stream of the face of the patient, in order to measure the respiratory rate and the cardiac rate of the patient. In the illustrated example, the system is placed at a distance from the face of the patient equal or smaller than one meter.

The system is able to detect on said image stream predefined facial cues, or expressions, on the face of the patient associated to respiratory distress, an action score being attributed based on the presence or absence of said facial cues. A respiratory score is also attributed based on the measured value of the respiratory rate, as well as a cardiac score based on the measured value of the cardiac rate.

A global score is then computed by adding said action, respiratory and cardiac scores, and, if said global score is greater than a predefined threshold, an action, an alarm in this example, is triggered to indicate respiratory distress. A caregiver may then enter the room and interact with the patient, and therapeutic measures may be undertaken in order to relieve the respiratory suffering.

In this example and preferably, said predefined facial cues are facial action units, chosen among frowning, internal raising of the eyebrows, external raising of the eyebrows, and widening of the eyelids.

The system may further include at least one learning module, trained beforehand to learn reference facial cues on previously acquired image streams from patients, said learning module being configured to detect said facial cues on the image stream from the patient under monitoring.

In this example, the measurement of the respiratory and cardiac rates is averaged over a period of 10 seconds, giving six values per minute, said respiratory and cardiac scores being computed by summing the mean values of the measured respiratory and cardiac rates and by adding one point to the action score each time a facial cue is detected.

Preferably, the acquisition device further includes a thermal camera. The image stream from the thermal camera and the image stream from the video camera may then be merged. In this embodiment, the thermal camera has a spectral range comprised between 7.5 μm and 14 μm, and an optimal thermal sensitivity equal to 50 mK at 30 °C.

In this example and preferably, the system is configured so that the values of the respiratory rate are obtained from measures of the temperature variations of the artificial ventilation circuit, and the values of the cardiac rate are obtained from measures of temperature variations on the surface of the face of the patient, for example the cheeks or forehead.

The invention is not limited to the examples that have just been described.

For example, different ways of computing the different scores may be used in the system and method according to the invention.

The invention may be used to detect dyspnea or respiratory distress in resuscitation patients undergoing invasive mechanical ventilation who are unable to report their symptoms. Because of the intubation, more than 50% of intubated resuscitation patients are unable to communicate their symptoms/suffering to caregivers. The invention would therefore affect a large number of patients.

The invention, when based on thermal sensing signals, would allow detection of dyspnea also in low light conditions such as at night.

An extension of the field of application may be imagined, for example in conventional medicine for non-intubated patients, especially in pneumology, or in telemedicine at home to detect acute dyspnea in patients.

1. A system for monitoring respiratory suffering or discomfort on a patient, placed under artificial ventilation in an intensive care unit, the system comprising:
an acquisition device including at least a video camera and configured to acquire an image stream of the face of said patient,
the system being configured to measure the respiratory rate of said patient and the cardiac rate of said patient, the system being configured to:
detect on said image stream at least one predefined facial cue on the face of said patient associated to respiratory distress, an action score being attributed based on the presence or absence of said facial cue(s),
attribute a respiratory score based on the measured value of the respiratory rate,
attribute a cardiac score based on the measured value of the cardiac rate,
compute a global score by adding said action, respiratory and cardiac scores, and
if said global score is greater than a predefined threshold, trigger an action, especially an alarm, to indicate respiratory distress.

2. The system of claim 1, wherein said predefined facial cues are facial action units, selected from the group consisting of: frowning, internal raising of the eyebrows, external raising of the eyebrows, and widening of the eyelids.

3. The system of claim 1, wherein said acquisition device further includes a thermal camera.

4. The system of claim 1, said patient being placed under artificial ventilation in an intensive care unit, the system being configured so that the values of the respiratory rate are obtained from measures of the temperature variations of the artificial ventilation circuit.

5. The system of claim 1, being configured so that the values of the cardiac rate are obtained from measures of temperature variations on the surface of the face of the patient, especially the cheeks or forehead.

6. The system of claim 1, being further configured to measure the r transcutaneous oxygen saturation of said patient, and to attribute an oxygenation score based on said measured value of transcutaneous oxygen saturation.

7. The system of claim 3, wherein the image stream from the thermal camera and the image stream from the video camera are merged.

8. The system of claim 1, wherein said system further includes at least one learning module, trained beforehand to learn reference facial cues on previously acquired image streams from patients, said learning module being configured to detect said facial cues on the image stream from the patient under monitoring.

9. The system of claim 1, being placed at a distance from the face of the patient equal or smaller than one meter.

10. The system of claim 1, wherein the measurement of the respiratory and cardiac rates is averaged over a period of 10 seconds, giving six values per minute, said respiratory and cardiac scores being computed by summing the mean values of the measured respiratory and cardiac rates and by adding one point to the action score each time a facial cue is detected.

11. The system of claim 3, wherein said thermal camera has a spectral range comprised between 7.5 μm and 14 μm.

12. The system of claim 3, wherein said thermal camera has an optimal thermal sensitivity equal to 50 mK at 30 °C.

13. A method for monitoring respiratory suffering or discomfort on a patient, placed under artificial ventilation in an intensive care unit, using a system comprising an acquisition device including at least a thermal camera and configured to acquire an image stream of the face of said patient, the system being configured to measure the respiratory rate of said patient, and to measure the cardiac rate of said patient,
the method comprising at least the steps of:
detecting on said image stream at least one predefined facial cue on the face of said patient associated to respiratory distress, an action score being attributed based on the presence or absence of said facial cue(s),
attributing a respiratory score based on the measured value of the respiratory rate,
attributing a cardiac score based on the measured value of the cardiac rate,
computing a global score by adding said action, respiratory and cardiac scores, and
if said global score is greater than a predefined threshold, triggering an action, especially an alarm, to indicate respiratory distress.

14. The method of claim 13, using at least one learning module, trained beforehand to learn reference facial cues on previously acquired image streams from patients, the method comprising the step of using said learning module to detect said facial cues on the image stream from the patient under monitoring.

15. A computer program comprising instructions which, when the program is executed on a computer, cause the computer to carry out the steps of the method according to claim 13.
